(19) United States
(12) Patent Application Publication
(10) Pub. No.: US 2016/0367502 A1
(43) Pub. Date: Dec. 22, 2016

(54) FILM COATED PELLETS

(71) Applicant: RATIOPHARM GMBH, Ulm (DE)

(72) Inventors: Max-Werner SCHEIWE, Maulburg (DE); Dieter SWATSCHEK, Blaubeuren (DE); Isabel STEINHAUSER, Frankfurt/Main (DE)

(21) Appl. No.: 14/653,652

(22) PCT Filed: Dec. 19, 2013
(86) PCT No.: PCT/EP2013/077278

§ 371 Date: Jun. 18, 2015

Related U.S. Application Data
(60) Provisional application No. 61/739,046, filed on Dec. 19, 2012.

(30) Foreign Application Priority Data
Dec. 19, 2012 (EP) 12008457.9

(51) Int. Cl. A61K 31/137; A61K 9/48

(52) U.S. Cl. CPC A61K 31/137; A61K 9/4891; A61K 9/4816; A61K 9/4833

(57) ABSTRACT

The invention relates to film coated pellets comprising an active ingredient having a high solubility in water. The invention also relates to methods of preparing said pellets and to cores comprising the active ingredient having a high solubility in water and to pharmaceutical compositions comprising the film coated pellets.

The invention relates to starter pellets as well as uncoated or film coated cores comprising an active pharmaceutical ingredient (API) having a high solubility in water. The invention also relates to methods of preparing said starter pellets and said cores comprising the active pharmaceutical ingredient having a high solubility in water and to pharmaceutical compositions comprising the uncoated and/or film coated cores.

High solubility in water of an active ingredient may cause problems during manufacturing processes using evaporation because the active pharmaceutical ingredient can create a sticky film during processing when used in a solution below its saturation concentration at the process temperature. When coating inert seeds with such an active pharmaceutical ingredient this may lead to the formation of a sticky coating on the seeds which makes drug loading almost impossible because the resulting pellets stick together. The tendency to form a sticky coating may even vary between batches as small variances in API production may influence the physicochemical properties of the API, thus requiring a process that can be adapted easily without transgression of regulatory requirements. Moreover these pellets are difficult to handle in subsequent processing steps as they tend to form agglomerates and therefore have bad flowing properties. Additionally active pharmaceutical ingredients having a high solubility in water do not easily precipitate out of solution and accordingly cannot easily be coated on inert seeds in processes using evaporation like for example fluidized bed coating.

WO 2005/013939 relates to a process of coating inert seeds with an active pharmaceutical ingredient having a high solubility in water using a supersaturated solution of the active pharmaceutical ingredient containing a desired amount of the API in order to overcome the problems of poor precipitation and formation of a sticky coating when using an active pharmaceutical ingredient having a high solubility in water in manufacturing processes like fluidized bed coating.

The use of supersaturated solutions of an active pharmaceutical ingredient having a high solubility in water though is problematic as preliminary precipitation resulting from changes in the processing temperature may occur. This may lead to clogging of pipes, valves or nozzles of the production line ultimately resulting in interruption of the whole manufacturing process. Thus there is a need to provide a less sensitive manufacturing process for coating seeds with an active pharmaceutical ingredient having a high solubility in water while simultaneously avoiding the formation of a sticky coating on the seeds.

### SUMMARY OF THE INVENTION

It has now been surprisingly found that the use of seeds consisting of the pure compacted active pharmaceutical ingredient having a high solubility in water (compacted API seeds) instead of inert seeds allows for the use of an unsaturated solution of active pharmaceutical ingredient having a high solubility in water for coating the seeds, thus avoiding preliminary precipitation of the API in the production line due to changes in the processing temperature. It was also found that the use of supersaturated solutions of an active pharmaceutical ingredient having a high solubility in water is not advisable when using compacted API seeds as this may lead to API coated seeds having an uneven surface. This can be problematic for later coating steps as no homogenous coating film can be achieved on an uneven surface. Ultimately this may lead to differences in the release rate of the active pharmaceutical ingredient from the resulting film coated cores.

In contrast the use of compacted API seeds in combination with a unsaturated solution of the active pharmaceutical ingredient having a high solubility in water resulted in a optimal coating of the seeds with the active pharmaceutical ingredient such that a reproducible, homogenous and even coating is obtained. It was even more surprising that the compaction of the pure API without any excipients leads to compacted API seeds stable enough to be used as seeds in fluidized bed coating. Moreover compacted API seeds consisting of pure active pharmaceutical ingredient having a high solubility in water were stable enough to be used as seeds in fluidized bed coating even when water was used as solvent in the spraying solution.

It is thus a subject of the present invention to provide a method for the preparation of compacted API seeds by compacting an active ingredient having a high solubility in water. Another subject of the present invention are compacted API seeds obtainable by the aforementioned method.

Moreover the present process allows using an unsaturated solution of the active pharmaceutical ingredient having a high solubility in water without leading to poor precipitation of the API or the formation of a sticky coating on the seeds. In addition the use of compacted API seeds consisting of the pure active pharmaceutical ingredient alone allows for achieving a higher drug load in the final product compared to products of the same size having an inert core.

The above mentioned objects unexpectedly are thus achieved by the method as described in claim 1. Hence, a further subject of the present invention is a method for the preparation of starter pellets comprising an active pharmaceutical ingredient having a high solubility in water comprising the steps of

(1) preparing a powder mixture comprising compacted API seeds and one or more pharmaceutical excipients;
(2) preparing a spraying solution comprising the active pharmaceutical ingredient having a high solubility in water and a solvent and
(3) spraying the solution of step (2) on the powder mixture obtained in step (1) to obtain starter pellets.

Another subject of the present invention are the starter pellets obtainable by the above described method. Preferably the starter pellets have a particle size distribution of a D10-value of 500 to 1000 μm and/or a D50-value of 700 to 1100 μm.

In an additional subject the invention provides a method for the preparation of cores comprising an active pharmaceutical ingredient having a high solubility in water comprising the steps of
(a) preparing a powder feed comprising an active pharmaceutical ingredient having a high solubility in water and one or more pharmaceutical excipients;
(b) preparing a spraying solution comprising the active pharmaceutical ingredient having a high solubility in water and a solvent;
(c) preparing a mixture of powder feed and starter pellets by combining one part of the powder feed from step (a) with the starter pellets according to the present invention;
(d) spraying the solution of step (b) on the mixture from step (c) and
(e) repeating steps (c) and (d) until all of the powder feed of step (a) is admixed with the starter pellets to obtain cores.

In yet another subject the invention relates to a core obtainable by the above described method for the preparation of cores. Preferably the cores have a particle size distribution of a D10-value of 400 to 1000 μm and/or a D50-value of 750 to 1350 μm.

Moreover it is a subject of the present invention to provide a pharmaceutical composition comprising the cores according to the invention.

A further subject of the present invention is a method for the preparation of film coated cores comprising the steps of
(i) preparing a coating dispersion and
(ii) coating the cores according to the present invention with the coating dispersion (i) to obtain film coated cores.

Still another subject of the present invention are film coated cores obtainable by the method of the present invention. Moreover a further subject of the present invention is a pharmaceutical composition comprising the starter pellets, cores and/or film coated cores of the present invention.

### DETAILED DESCRIPTION OF THE INVENTION

Throughout this application the terms “active pharmaceutical ingredient having a high solubility in water” and “active pharmaceutical ingredient” are used synonymously in the sense that the term “active pharmaceutical ingredient” always means “active pharmaceutical ingredient having a high solubility in water” unless specifically denoted otherwise.

Preferably the active pharmaceutical ingredient having a high solubility in water is an active ingredient having a solubility in water at 25° C. of between 100 mg per gram of water to 10 gram per gram of water, such as between 300 mg per gram of water to 10 gram per gram of water at 25° C. and more preferably between 400 mg per gram of water to 10 gram per gram of water at 25° C. and most preferably between 500 mg per gram of water to 10 gram per gram of water at 25° C. According to USP definition such an active ingredient would be classified as freely soluble or very soluble in water (see Table 1.). Preferably the active pharmaceutical ingredient having a solubility in water at 25° C. of between 100 mg per gram of water to 10 gram per gram of water is freely soluble or very soluble in water as defined in USP.

TABLE 1
Values for estimating solubility of an active ingredient
based upon “USP definition”

| | Appropriate Volume of Solvent In Millilitres |
|---|---|
| Descriptive Term | Per Gram of Solute |
| Very soluble | Less than 1 part solvent needed to |
| | dissolve 1 part solute |
| Freely soluble | From 1 to 10 parts solvent needed to |
| | dissolve 1 part solute |
| Soluble | From 10 to 30 parts solvent needed to |
| | dissolve 1 part solute |
| Sparingly soluble | From 30 to 100 parts solvent needed to |
| | dissolve 1 part solute |
| Slightly soluble | From 100 to 1000 parts solvent needed to |
| | dissolve 1 part solute |
| Very slightly | From 1000 to 10,000 parts solvent needed to |
| soluble | dissolve 1 part solute |
| Practically | More than 10,000 parts solvent needed to |
| insoluble | dissolve 1 part solute |

The solubility in water at 25° C. of an active pharmaceutical ingredient is generally the solubility in water at 25° C. given in the European Pharmacopoeia. If the solubility in water at 25° C. for a given active pharmaceutical ingredient is not specified in the European Pharmacopoeia it is determined in the context of this invention by using the flask method as described in EU Regulation (EC) No 440/2008 Annex part A: Methods for the determination of physico-chemical properties Chapter A.6.

The term “active pharmaceutical ingredient having a high solubility in water” comprises the active ingredient in form of the free base as well as pharmaceutically acceptable salts, hydrates, co-crystals, solvates and/or polymorphs of an active ingredient if those pharmaceutically acceptable salts, hydrates, co-crystals, solvates and/or polymorphs have a high solubility in water.

Examples of active pharmaceutical ingredients having a high solubility in water include metoprolol tartarate, diltiazem salts, pseudoephedrine salts, phenyltolaxamine, brompheniramine maleat, diphenhydramine, vancomycin hydrochloride, tapentadol hydrobromide, sitagliptin salts such as sitagliptin dihydrogen phosphate, sitagliptin (D)-glucuronate, sitagliptin glutarate, sitagliptin hydrogen sulfate, sitagliptin (L)-lactate and sitagliptin oxalate, venlafaxine salts or desvenlafaxine salts and the like. Preferably the active pharmaceutical ingredient having a high solubility in water is a venlafaxine or desvenlafaxine salt. More preferred the active pharmaceutical ingredient having a high solubility in water is an active pharmaceutical ingredient having a solubility in water at 25° C. of greater than or equal to 100 mg per gram of water, such as a venlafaxine salt, preferably venalafaxine hydrogenmaleate anhydrate as described in WO 03/082805 A1 or venlafaxine HCl (see Table 2). Mostly preferred the active pharmaceutical ingredient having a solubility in water at 25° C. of between 100 mg per gram of water to 10 gram per gram of water is venlafaxine HCl. Venlafaxine is the common name for the compound 1-[2-(dimethylamino)-1-(4-methoxyphenyl) ethyl] cyclohexanol, having the structure shown below:

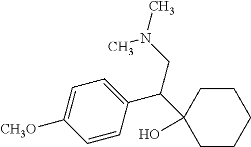

U.S. Pat. No. 4,535,186 describes a class of hydroxycycloalkanephenethyl amines as being useful antidepressants and exemplifies the compound now known as venlafaxine HCl as one of the suitable species in example 3. As stated above it is preferred that the active pharmaceutical ingredient having a high solubility in water is a venlafaxine salt, mostly preferred venlafaxine HCl, and particularly preferred a pure polymorphic form. Form I of venlafaxine HCl is defined as in article in Acta Crystallographica (2000) August; 56, 1009-10, various other polymorphs are disclosed in WO 02/45658, WO 02/36542, WO 02/46140, WO 03/042161, WO 03/048082, WO 03/050075 and WO 03/050074.

The most preferred polymorphic form of venlafaxine HCl is polymorphic form I. In another preferred embodiment venlafaxine HCl is the sole active pharmaceutical ingredient.

The active pharmaceutical ingredient having a high solubility in water preferably is used in a solid form. The solid form may be the crystalline or amorphous state or it may be a mixture of both crystalline and amorphous state.

TABLE 2
Solubility in water of venlafaxine salts at 25° C.

| | Solubility in distilled water |
|---|---|
| Venlafaxine Salt | at 25° C. in mg/g water |
| venlafaxine HCl | 572 |
| venlafaxine hydrogenmaleate | 368 |
| anhydrate | |

Alternatively particular preferred active pharmaceutical ingredient having a solubility in water at 25° C. of between 100 mg per gram of water to 10 gram per gram of water are sitagliptin salts such as sitagliptin dihydrogen phosphate, sitagliptin D-glucuronate, sitagliptin glutarate, sitagliptin hydrogen sulfate, sitagliptin L-lactate and sitagliptin oxalate.

TABLE 2a
Solubility of sitagliptin salts in water at 25° C.

| | Dihydrogen | D- | | Hydrogen | L- |
|---|---|---|---|---|---|
| 25° | phosphate | Glucuronate | Glutarate | sulfate | Lactate |
| mg/ml | 100-120 | >500 | >500 | >300 | >300 |
| (water) | | | | | |

The term “pure” when used throughout this application is to be understood in the way that no other substances, especially no excipients are added. In this sense the term “pure” means consisting of 95% to 100% of the according substance, more preferably consisting of 97% to 100%, even more preferably consisting of 99% to 100%, most preferably consisting of 99.5% to 100%, of the according substance.

The term “spraying solution” as used by the present invention relates to a solution comprising a solvent and an active pharmaceutical ingredient having a high solubility in water. Preferably the solvent is an aqueous solvent, such as water. The water that is used in dissolving the active pharmaceutical ingredient is water that is normally used in the pharmaceutical arts. It may be tap water or spring water, but it is preferred that the water is deionized or distilled water. It is also preferred, that the active pharmaceutical ingredient is present in a concentration below its saturation concentration at 25° C. in the solvent. The spraying solution may comprise equal to or less than 50% (w/w) active pharmaceutical ingredient, such as equal to or less than 45% (w/w), for example equal to or less than 40% (w/w), such as equal to or less than 35% (w/w), for example equal to or less than 30% (w/w) of active pharmaceutical ingredient. For example if the active pharmaceutical ingredient is venlafaxine HCl and the solvent is water, the spraying solution preferably comprises equal to or less than 30% (w/w) venlafaxine HCl. Preferably the spraying solution may comprise between 10% to 50% (w/w) active pharmaceutical ingredient, such as between 15% to 40% (w/w), for example between 15% to 35% (w/w), such as between 15% to 30% (w/w) of active pharmaceutical ingredient. Preferably the active pharmaceutical ingredient having a high solubility in water is an active pharmaceutical ingredient having a solubility in water at 25° C. of greater than or equal to 100 mg per gram of water. More preferred the active pharmaceutical ingredient having a solubility in water at 25° C. of between 100 mg per gram of water to 10 gram per gram of water is a venlafaxine salt, such as venlafaxine hydrogenmaleate anhydrate or venlafaxine HCl. Mostly preferred the active pharmaceutical ingredient having a solubility in water at 25° C. of between 100 mg per gram of water to 10 gram per gram of water is venlafaxine HCl. It is also preferred that the spraying solution does not contain a binder. Mostly preferred the spraying solution consists of the solvent and a pure active pharmaceutical ingredient having a high solubility in water. Even more preferred the spraying solution consists of a solvent and venlafaxine HCl.

Each drug has its own saturation concentration in water at 25° C. If it is not known, it can be determined by conventional methods. For example, a known amount of the active pharmaceutical ingredient having a high solubility in water is added to a container of known weight, containing 1 gram of water as solvent at a desired temperature, e. g. 25° C. As step by step a known amount of the active pharmaceutical ingredient having a high solubility in water is added to the water, there will be a point when no more active pharmaceutical ingredient will dissolve. At this point, the water cannot hold any more active pharmaceutical ingredient, and the excess active ingredient will fall to the bottom of the container and remain as a solid. The excess solid is separated from the water by techniques known in the art, such as by decanting, filtering and the like; the separated solution contains the saturated solution of the active pharmaceutical ingredient at the desired temperature, for example 25° C. The amount or concentration of the active pharmaceutical ingredient in the solution is determined by analytical techniques known in the art. For example, if a known amount of active pharmaceutical ingredient is added to the water and the amount (dry weight) of the active pharmaceutical ingredient that is in excess is subtracted out, the difference is the amount of active pharmaceutical ingredient soluble in the solvent (i. e. water) at the given temperature. Thus, the saturation concentration is the weight percentage of the active pharmaceutical ingredient having a high solubility in water dissolved in the solvent (i.e. water) to the total weight of active pharmaceutical ingredient having a high solubility in water dissolved in the solvent (i.e. water) plus the weight of the solvent that was used (1 gram of water in the above described example).

As indicated hereinabove, the amount of active pharmaceutical ingredient having a high solubility in water used in preparing the spraying solution of the present invention is less than the saturation concentration of the active pharmaceutical ingredient at 25° C.

The “pharmaceutical composition” according to this invention may be delivered by any suitable rout. Preferably the pharmaceutical composition is delivered orally in the form of oral dosage forms. The oral dosage forms may be single unit dosage forms (SUDFs), such as monolithic tablets, troches, lozenges or capsules, or multiple unit dosage forms (MUDFs), such as multi unit particulate tablets, multi unit pellet system (MUPS), granules/pellets/cores/mini-tablets filled into capsules, sachets, stick packs and other dosage forms suitable for oral administration. Preferably the pharmaceutical composition comprising the starter pellets, cores and/or film coated cores of the present invention is in form of pellets, cores and/or film coated cores filled into capsules. The pharmaceutical composition may be a pharmaceutical composition for immediate release or a pharmaceutical composition for modified release. Preferably the pharmaceutical composition is a pharmaceutical composition for modified release, such as delayed release, prolonged release, sustained release, repeated action release, extended release, controlled release and/or targeted release. More preferred the pharmaceutical composition is a composition for extended release.

The term “modified release” is used as defined by the USP. Preferably, modified release dosage forms are those whose release characteristics of the active pharmaceutical ingredient accomplish therapeutic or convenience objectives not offered by immediate release forms. Generally, immediate release (IR) forms release at least 80% of the active pharmaceutical ingredient within 1 hour or less. The term “modified release” can comprise delayed release, prolonged release, sustained release, repeated action release, extended release, controlled release and/or targeted release. Extended release is preferred.

Delayed release usually indicates that the active pharmaceutical ingredient (i.e., venlafaxine HCl) is not being released immediately after administration but at a later time, preferably less than 10% are released within two hours after administration. Prolonged release usually indicates that the active pharmaceutical ingredient (i.e., venlafaxine HCl) is provided for absorption over a longer period of time than IR forms, preferably for about 2 to 24 hours, in particular for 3 to 12 hours. Sustained release usually indicates an initial release of the active pharmaceutical ingredient (i.e., venlafaxine HCl), sufficient to provide a therapeutic dose soon after administration, preferably within two hours after administration, and then a gradual release after an extended period of time, preferably for about 3 to 18 hours, in particular for 4 to 8 hours. Repeated action release is distinguished from a sustained release by the fact that the release of the second dose is released at a later time point separated from the initial dose in contrast to a gradual release.

Extended release usually indicates a slow release of the active pharmaceutical ingredient (i.e., venlafaxine HCl), so that plasma concentrations are maintained at a therapeutic level for a time period of between 6 and 36 hours, preferably between 8 and 24 hours.

Controlled release dosage forms usually release the active pharmaceutical ingredient (i.e., venlafaxine HCl) at a constant rate and provide plasma concentrations that remain essentially invariant with time.

In a preferred embodiment, the oral dosage form of the present invention is an extended release oral dosage form.

In particular, the oral dosage form of the present invention shows a release of active pharmaceutical ingredient of less than 20% within 2.0 hours. Further, the oral dosage form of the present invention shows a release of active pharmaceutical ingredient of more than 35% within 3.0 to 12.0 hours, preferably between 4.0 and 8.0 hours.

Generally, within this application for oral dosage forms having an enteric coating the release profile is determined according to USP 31-NF26 release method, apparatus II (paddle). Dissolution media (0.1N HCl, Phosphate buffer pH 6.8, Acetate buffer pH 4.5, or 0.5% SLS in water) is selected on the basis of the active ingredient in the composition. For example if the active ingredient is venlafaxine HCl Phosphate buffer pH 6.8 is used. The measurements are carried out in preferably 900 ml 0.1 N HCl at 37° C., wherein the stirring speed was 75 rpm, and re-buffering after 2 hours to pH 6.8. For oral dosages forms not having an enteric coating generally the following protocol is applied: the release profile is determined according to USP 31-NF26 release method, apparatus I (basket). Dissolution media (0.1N HCl, phosphate buffer pH 6.8, Acetate buffer pH 4.5, or 0.5% SLS in water) is selected on the basis of the active ingredient in the composition. For example if the active ingredient is venlafaxine HCl phosphate buffer pH 6.8 is used. The measurements are carried out in preferably 500 ml phosphate buffer pH 6.8 at 37° C., wherein the stirring speed was 75 rpm.

Targeted release dosage forms deliver the active pharmaceutical ingredient in a manner that increases the concentration of the active pharmaceutical ingredient in some parts of the body relative to others. Preferably the active pharmaceutical ingredient is released at/or near the site of the diseased tissue. Targeted release dosage forms may have extended release characteristics. For example the active pharmaceutical ingredient may be targeted to the site of action by the use of antibodies.

“Particle diameter” or “particle size” of a particle to be determined means according to the invention the diameter of an equivalent particle which is assumed to be spherical and to have the same light scattering pattern as the particle to be determined. According to the invention, particle size is determined by means of laser diffractometry. More specifically, the particle size was determined using a Mastersizer 2000 from Malvern Instruments. The particle size determination may be carried out as a wet or a dry measurement depending on the sample. If a wet measurement is used the particles of which the particle size is to be determined are dispersed in a dispersant, preferably a dispersant having a RI of 1.000 and the measurement is carried out at 1750 rpm and ultrasound for 30 s.

Preference is given to carrying out a dry measurement using a Mastersizer 2000 from Malvern Instruments and a Scirocco 2000 dry powder feeder and a gaseous dispersant. In a preferred embodiment the following settings are used:

| Beam length | 10.00 | mm |
|---|---|---|
| Slit width | 8 | mm |
| Feed rate | 60% | |
| Dispersive air pressure | 1.5 | bar |
| Sample volume | 9 | ml |
| Number of measurement Cycle | 1 | |
| Sample Measurement time | 65.5 | sec |
| Measurement integration time | 65500 | ms |

| | |
|---|---|
| Measuring range | 0.020 to 2000.000 μm |
| Dispersant RI | 1.000 |

Particles with a D 50 value of less than 5.0 μm are evaluated with the aid of the Mie method, and particles with a D 50 value of 5.0 μm or larger are evaluated with the aid of the Fraunhofer method.

“Particle size distribution” here means the statistical distribution of the partial volumes based on all available particle sizes of the sample measured. “Partial volume” means the volume-based percentage of all particles having a defined particle size. According to the invention, the particle size distribution D 50 value describes the particle size at which 50% by volume of the particles have a smaller particle size than the particle size corresponding to the D 50 value. Likewise, 50% by volume of said particles then have a larger particle size than the D 50 value.

Accordingly, the D90 value of the particle size distribution of the compacted API seeds, starter pellets, cores or film coated cores is defined as the particle size at which 90% by volume of the particles have a smaller particle size than the particle size corresponding to the D90 value. Similarly, the D10 value of the particle size distribution of the compacted API seeds, starter pellets, cores or film coated cores is defined as the particle size at which 10% by volume of the particles have a smaller particle size than the particle size corresponding to the D 10 value.

The term “powder mixture” according to this invention refers to a mixture comprising compacted API seeds and one or more pharmaceutical excipients. The powder mixture preferably comprises 30% to 95% (w/w) of compacted API seeds based on the total weight of the powder mixture, such as between 35 to 85% (w/w) of compacted API seeds based on the total weight of the powder mixture, preferably between 45 to 75% (w/w) of compacted API seeds based on the total weight of the powder mixture, more preferably between 55 to 65% (w/w) of compacted API seeds based on the total weight of the powder mixture.

Preferably the powder mixture comprises between 5% to 70% (w/w) of excipients based on the total weight of the powder mixture, such as between 15% to 65% (w/w) of excipients based on the total weight of the powder mixture, preferably between 25% to 55% (w/w) of excipients based on the total weight of the powder mixture, more preferably between 35% to 45% (w/w) of excipients based on the total weight of the powder mixture.

Preferably the pharmaceutical excipients are in the form of dry components.

The term “powder feed” when used in the context of this invention refers to mixture comprising an active pharmaceutical ingredient having a high solubility in water and one or more pharmaceutical excipients. The powder feed preferably comprises 30% to 95% (w/w) of active pharmaceutical ingredient having a high solubility in water based on the total weight of the powder feed, such as between 40% to 80% (w/w) of active pharmaceutical ingredient having a high solubility in water based on the total weight of the powder feed, preferably between 40% to 70% (w/w) of active pharmaceutical ingredient having a high solubility in water based on the total weight of the powder feed, more preferably between 40% to 60% (w/w) of active pharmaceutical ingredient having a high solubility in water based on the total weight of the powder feed, most preferably between 40% to 50% (w/w) of active pharmaceutical ingredient having a high solubility in water based on the total weight of the powder feed.

Preferably the powder feed comprises between 5% to 70% (w/w) of excipients based on the total weight of the powder feed, such as between 20% to 60% (w/w) of excipients based on the total weight of the powder feed, preferably between 30% to 60% (w/w) of excipients based on the total weight of the powder feed, more preferably between 40% to 60% (w/w) of excipients based on the total weight of the powder feed, most preferably between 50% to 60% (w/w) of excipients based on the total weight of the powder feed.

Preferably the active pharmaceutical ingredient having a high solubility in water is an active pharmaceutical ingredient having a solubility in water at 25° C. of greater than or equal to 100 mg per gram of water. More preferred the active pharmaceutical ingredient having a solubility in water at 25° C. of between 100 mg per gram of water to 10 gram per gram of water is a venlafaxine salt, such as venlafaxine hydrogenmaleate anhydrate or venlafaxine HCl. Mostly preferred the active pharmaceutical ingredient having a solubility in water at 25° C. of between 100 mg per gram of water to 10 gram per gram of water is venlafaxine HCl.

Preferably the pharmaceutical excipients are in the form of dry components.

The term” coating film” relates to a film comprising on or more polymers. The coating film is applied to the cores of the present invention by coating the cores with the coating dispersion as described in the method for the preparation of film coated cores of the present invention. The polymers can either be polymers modifying the release of the active pharmaceutical ingredient or polymers not modifying the release of the active pharmaceutical ingredient. Preferably the polymers are modified release polymers, such as delayed release, prolonged release, sustained release, repeated action release, extended release and/or controlled release polymers. More preferred the modified release polymers are selected from the group consisting of cellulosic polymers, methacrylic acid polymers and waxes. Mostly preferred the modified release polymers are selected from the group consisting of ethyl cellulose, hydroxypropyl methylcellulose, hydroxypropyl cellulose, methylcellulose, carboxymethylcellulose, hydroxymethylcellulose, and hydroxyl-ethylcellulose, hydroxypropylmethyl phthalate, cellulose acetate phthalate, and cellulose acetate trimellitate. Particularly preferred, ethyl cellulose is used as modified release polymer. Ethyl cellulose can, for example, be used in the form of the commercially available Aquacoat® ECD system (FMC BioPolymer, approximately 24.5% to 29.5% ethyl cellulose in aqueous solution). If the coating film comprises modified release polymers, such as delayed release, prolonged release, sustained release, repeated action release, extended release and/or controlled release polymers the according coating film is a modified release coating film, such as a delayed release, prolonged release, sustained release, repeated action release, extended release and/or a controlled release coating film.

Preferred examples of polymers, which do not affect the release of the active pharmaceutical ingredient, can be those including polyvinyl alcohol, starch, modified starch, polyvinylpyrrolidone, sugars, sugar alcohols, aminoalkyl-methacrylate-copolymers, carbohydrates such as maltodextrins and polydextrose, mixtures of microcrystalline cellulose with carrageenan and the like.

The coating film may comprise one or more pharmaceutically excipients and additives like e. g., film forming agent, anti-foam agent, filler, coloring agent, flavoring agent, perfumes, sweetening agent, surface active agent, lubricant, stabilizing agent, antiadherent, pore former, plasticizer and the like, or mixtures thereof.

In one embodiment the present invention provides a method for the preparation of compacted API seeds by compacting an active pharmaceutical ingredient having a high solubility in water. Preferably pure active pharmaceutical ingredient is compacted. Preferably the active pharmaceutical ingredient having a high solubility in water is an active pharmaceutical ingredient having a solubility in water at 25° C. of greater than or equal to 100 mg per gram of water. More preferred the active pharmaceutical ingredient has a solubility in water at 25° C. of between 100 mg per gram of water to 10 gram per gram of water. Preferably the active pharmaceutical ingredient having a solubility in water at 25° C. of between 100 mg per gram of water to 10 gram per gram of water is a venlafaxine salt, such as venlafaxine hydrogenmaleate anhydrate or venlafaxine HCl, or desvenlaflaxine (=4-[2-dimethylamino-1-(1-hydroxycyclohexyl)ethyl]phenol), preferably venlaflaxine or desvenlaflaxine. Mostly preferred the active pharmaceutical ingredient having a solubility in water at 25° C. of between 100 mg per gram of water to 10 gram per gram of water is venlafaxine HCl.

The compacting can be carried out by any method known in the art such as dry compaction or slugging. The equipment used in these methods is known in the art. For example dry compaction can be carried out by using a roller compactor while for slugging a state of the art tablet press may be used.

Optionally the compacted API seeds may be sieved.

Preferably the compaction process is carried out in such a way that the resulting compacted API seeds have a particle size of 0.1 to 2.00 mm, preferably of 0.2 to 1.5 mm, such as 0.4 to 1.00 mm.

The compaction force used depends on the active pharmaceutical ingredient and has to be in a range that will produce compacted API seeds stable enough to be used as seeds in fluidized bed coating, such as in a range between 5 to 50 kN/cm. In a preferred embodiment the compaction force can be between 6 to 50 kN/cm, preferably between 8 to 50 kN/cm, more preferably between 10 to 40 kN/cm, even more preferably 12 to 35 kN/cm, in particular 15 to 25 kN/cm.

For example if the active pharmaceutical ingredient having a high solubility in water is venlafaxine HCl the compaction force preferably is between 10 to 30 kN/cm, such as between 15 to 20 kN/cm.

Another embodiment of the present invention are compacted API seeds obtainable by the aforementioned methods. The compacted API seeds of the present invention consist of compacted active pharmaceutical ingredient having a high solubility in water. Preferably the compacted API seeds consist of compacted active pharmaceutical ingredient having solubility in water at 25° C. of greater than or equal to 100 mg per gram of water. More preferred the compacted API seeds consist of compacted venlafaxine salt, such as venlafaxine hydrogenmaleate anhydrate or venlafaxine HCl, or desvenlaflaxine. Mostly preferred the compacted API seeds consist of compacted venlafaxine HCl. Preferably the compacted API seeds consist of the pure active pharmaceutical ingredient. The compacted API seeds of the present invention preferably have a particle size of 0.1 to 2.00 mm, more preferably of 0.2 to 1.5 mm, such as 0.4 to 1.00 mm.

In a further embodiment the present invention provides a method for the preparation of starter pellets comprising an active pharmaceutical ingredient having a high solubility in water comprising the steps of
(1) preparing a powder mixture comprising compacted API seeds and one or more pharmaceutical excipients;
(2) preparing a spraying solution comprising an active pharmaceutical ingredient having a high solubility in water and a solvent and
(3) spraying the solution of step (2) on the powder mixture obtained in step (1) to obtain starter pellets.

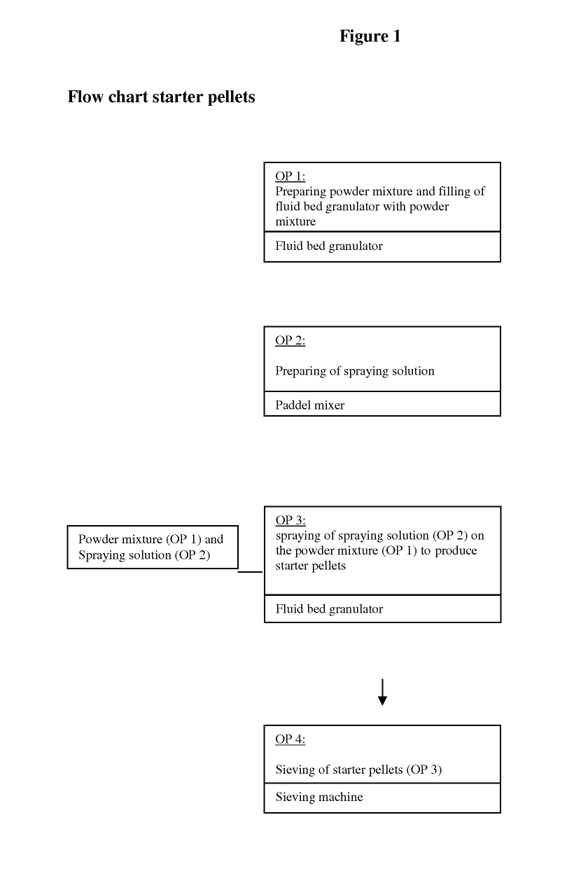

Optionally the method for the preparation of starter pellets may comprise further processing steps. For example the starter pellets obtained in step (3) may further be sieved. FIG. 1 shows an exemplary flow chart of the method for the preparation of starter pellets.

Mostly preferred the method for the preparation of starter pellets comprising an active pharmaceutical ingredient having a high solubility in water comprises the steps of
(1) preparing a powder mixture comprising compacted API seeds and one or more pharmaceutical excipients;
(2) preparing a spraying solution comprising an active pharmaceutical ingredient having a high solubility in water and a solvent;
(3) spraying the solution of step (2) on the powder mixture obtained in step (1) to obtain starter pellets and
(4) sieving the starter pellets from step (3).

The starter pellets obtained in any one of steps (3) and/or (4) can be compressed into monolithic tablets, multi unit particulate tablets, multi unit pellet system (MUPS) or filled into capsules.

Preferably the method for the preparation of starter pellets is carried out in such a way that the resulting starter pellets have a particle size distribution of a D10-value of 500 μm to 1000 μm and/or a D50-value of 700 μm to 1100 μm. More preferred the D10 is between 500 μm to 900 μm, such as between 600 μm to 800 μm. More preferred the D50 is between 800 μm to 1000 μm.

The above method is a conventional method that may be carried out in any suitable apparatus. For example a fluid bed apparatus, a pan coating apparatus or a rotor granulator may be used to carry out step (3) of the method for the preparation of starter pellets. It is preferred that a fluid bed apparatus is used such as for example a fluid bed granulator.

The method for the preparation of starter pellets according to the present invention is suitably performed as follows: Preferably the inlet air temperature of the fluid bed apparatus is between 30° C. to 70° C., such as between 40° C. to 70° C., preferably between 50° C. to 70° C., mostly preferred between 60° C. to 70° C.

Preferably the outlet temperature is between 20° C. to 45° C., such as between 30° C. to 45° C., mostly preferred between 35° C. to 40° C.

Preferably the product temperature is between 20° C. to 45° C., such as between 20° C. to 35° C., mostly preferred between 25° C. to 35° C.

Preferably the one or more pharmaceutical excipients in step (1) are separately selected from the group consisting of: film forming agent, thickening agent, filler, alkalizing agent, buffering agent, emulsifying agent, complexing agent, emulsion stabilizer, glidant, suspending agent, adsorbent, antiadherent, lubricant, disintegrant, pH-regulator, plasticizer, flow aid and binder. Such excipients are known to the person skilled in the art (see for example “Handbook of Pharmaceutical Excipients”, 3^{rd} edition, edited by Arthur H. Kibbe, American Pharmaceutical Association, Washington, USA and Pharmaceutical Press, London).

Preferably the binder is selected from the group consisting of acacia, alginic acid, carbomer, carboxymethylcellulose sodium, dextrin, ethylcellulose, gelatin, liquid glucose, guar gum, hydroxyethyl cellulose, hydroxypropyl cellulose, hydroxypropyl methylcellulose, magnesium aluminum silicate, maltodextrin, methylcellulose, polymethacrylates, sodium alginate, starch, pregelatinized starch, zein and/or povidone. Mostly preferred the binder is povidone.

Preferably the filler is selected from the group consisting of calcium carbonate, dibasic calcium phosphate dihydrate, tribasic calcium phosphate, calcium sulfate, powdered cellulose, dextrates, dextrin, dextrose, fructose, kaolin, magnesium aluminum silicate, magnesium carbonate, magnesium oxide, maltodextrin, mannitol, methylcellulose, microcrystalline cellulose, polymethacrylates, potassium chloride, sodium chloride, sorbitol, starch, pregelatinized starch, sucrose and/or lactose. Mostly preferred the filler is lactose and/or microcrystalline cellulose.

Preferably the pH-regulator is selected from the group consisting of potassium citrate, dibasic sodium phosphate, monobasic sodium phosphate, acetic acid, citric acid, sodium acetate, potassium phosphate, potassium hydrogen phthalate and/or sodium hydrogen citrate. Mostly preferred the filler is sodium hydrogen citrate.

Preferably the flow aid is selected from the group consisting of talc, siliconised talc, calcium stearate, magnesium stearate, aluminum stearate, stearic acid, starch, stearyl alcohol, cetyl alcohol, myristyl alcohol, polyethylene glycol, palmitic acid and/or colloidal silicon dioxide. Mostly preferred the filler is colloidal silicon dioxide.

Preferably the plasticizer is selected from the group consisting of triethyl citrate, acetyl triethyl citrate, tributyl citrate, acetyl tributyl citrate, dibutyl phathalate, diethyl phthalate, dimetyhl phathalate, glycerol triacetate, glycerol, polyethylene glycol, propylene glycol, destilled acetylated monoglycerides, castor oil, chlorobutanol and/or dibutyl sebacate. Mostly preferred the filler is dibutyl sebacate.

In this regard it is generally noted that due to the nature of pharmaceutical excipients it cannot be excluded that a certain compound meets the requirements of more than one of binder, filler, pH-regulator, flow aid or plasticizer. For example, in the present dosage form dextrin may act both as binder and as filler.

However, in order to enable an unambiguous distinction, it is preferred in the present application that one and the same pharmaceutical excipients can only function as one of binder, filler, pH-regulator, flow aid or plasticizer. For example, if dextrin is added as binder, it cannot additionally be added as filler.

The active pharmaceutical ingredient having a high solubility in water that is used in the method for the preparation of compacted API seeds and the active pharmaceutical ingredient having a high solubility in water that is used to prepare the spraying solution in step (2) of the method for the preparation of starter pellets may be the same or a different active pharmaceutical ingredient. Preferably the active pharmaceutical ingredient having a high solubility in water that is used in the method for the preparation of compacted API seeds and the active pharmaceutical ingredient having a high solubility in water that is used to prepare the spraying solution in step (2) of the method for the preparation of starter pellets is the same. More preferred the active pharmaceutical ingredient having a high solubility in water that is used in the method for the preparation of compacted API seeds and the active pharmaceutical ingredient having a high solubility in water that is used to prepare the spraying solution in step (2) of the method for the preparation of starter pellets is venlafaxine HCl.

In a further embodiment the invention relates to the starter pellets obtainable by the above described method to produce starter pellets. Preferably the starter pellets have a particle size distribution of a D10-value of 500 μm to 1000 μm and/or a D50-value of 700 μm to 1100 μm. More preferred the D10 is between 500 μm to 900 μm, such as between 600 μm to 800 μm. More preferred the D50 is between 800 μm to 1000 μm.

In yet another embodiment the invention discloses a method for the preparation of cores comprising an active pharmaceutical ingredient having a high solubility in water comprising the steps of
(a) preparing a powder feed comprising an active pharmaceutical ingredient having a high solubility in water and one or more pharmaceutical excipients;
(b) preparing a spraying solution comprising an active pharmaceutical ingredient having a high solubility in water and a solvent;
(c) preparing a mixture of powder feed and starter pellets by combining one part of the powder feed from step (a) with the starter pellets according to the invention;
(d) spraying the solution of step (b) on the mixture from step (c);
(e) adding a further part of the powder feed from step (a) to the product of step (d);
(f) spraying the solution of step (b) on the product from step (e) and
(g) repeating steps (e) and (f) until all of the powder feed of step (a) is admixed with the starter pellets according to the invention to obtain cores.

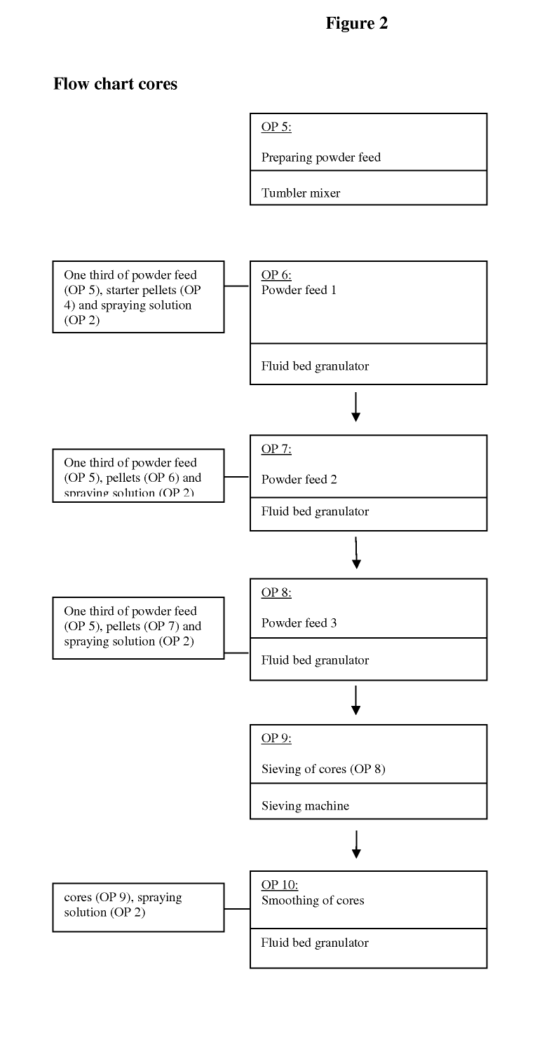

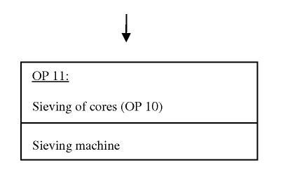

Optionally the method for the preparation of cores may comprise further processing steps. For example the cores obtained in step (g) may further be sieved and/or smoothened FIG. 2 shows an exemplary flow chart of the method for the preparation of cores. Preferably the smoothening is carried out by spraying the cores with the spraying solution of step (b).

Mostly preferred the method for the preparation of cores comprising an active pharmaceutical ingredient having a high solubility in water comprises the steps of
(a) preparing a powder feed comprising an active pharmaceutical ingredient having a high solubility in water and one or more pharmaceutical excipients;
(b) preparing a spraying solution comprising an active pharmaceutical ingredient having a high solubility in water and a solvent;
(c) preparing a mixture of powder feed and starter pellets by combining one part of the powder feed from step (a) with the starter pellets according to the invention;
(d) spraying the solution of step (b) on the mixture from step (c);
(e) adding a further part of the powder feed from step (a) to the product of step (d);
(f) spraying the solution of step (b) on the product from step (e) and
(g) repeating steps (e) and (f) until all of the powder feed of step (a) is admixed with the starter pellets according to the invention to obtain cores.
(h) sieving the cores from step (g);
(i) smoothen the cores from step (h) and
(j) sieving the cores from step (i).

The cores obtained in any one of steps (g), (h), (i) and or (j) can be compressed into monolithic tablets, multi unit particulate tablets, multi unit pellet system (MUPS) or filled into capsules.

Preferably the method for the preparation of cores is carried out in such a way that the resulting cores have a particle size distribution of D10 of 400 μm to 1000 μm and/or a D50 of 750 μm to 1350 μm. More preferred the D10 is between 500 μm to 900 μm, such as between 600 μm to 800 μm, mostly preferred between 650 μm to 800 μm. More preferred the D50 is between 800 μm to 1300 μm, such as between 900 μm to 1200 μm, mostly preferred between 950 μm to 1150 μm.

Preferably the method for the preparation of cores is carried out in such a way that the active pharmaceutical ingredient having a high solubility in water is present in an amount of 35% to 95% based on total weight of the core, such as between 45% to 95% based on total weight of the core, for example between 55% to 95% based on total weight of the core, mostly preferred between 65% to 95% based on total weight of the core.

The above method is a conventional method that may be carried out in any suitable apparatus. For example a fluid bed apparatus or a pan coating apparatus may be used to carry out steps (c) to (e) of the method for the preparation of cores. It is preferred that a fluid bed apparatus is used such as for example a fluid bed granulator.

The method for the preparation of cores according to the present invention is suitably performed as follows: Preferably the inlet air temperature of the fluid bed apparatus is between 30° C. to 75° C., such as between 40° C. to 70° C., preferably between 50° C. to 65° C., mostly preferred between 55° C. to 65° C.

Preferably the outlet temperature is between 20° C. to 45° C., such as between 30° C. to 45° C., mostly preferred between 35° C. to 40° C.

Preferably the product temperature is between 20° C. to 45° C., such as between 20° C. to 35° C., mostly preferred between 25° C. to 35° C.

Preferably the one or more pharmaceutical excipients in step (a) are separately selected from the group consisting of: film forming agent, thickening agent, filler, alkalizing agent, buffering agent, emulsifying agent, complexing agent, emulsion stabilizer, glidant, suspending agent, adsorbent, antiadherent, lubricant, disintegrant, plasticizer, pore former and binder.

The active pharmaceutical ingredient having a high solubility in water of the compacted API seeds, the active pharmaceutical ingredient having a high solubility in water that is used to prepare the spraying solution in step (2) of the method for the preparation of starter pellets and the active pharmaceutical ingredient having a high solubility in water that is used in steps (a) and/or (b) of the method for the preparation of cores may be the same or a different active pharmaceutical ingredient. Preferably the active pharmaceutical ingredient having a high solubility in water of the compacted API seeds, the active pharmaceutical ingredient having a high solubility in water that is used to prepare the spraying solution in step (2) of the method for the preparation of starter pellets and the active pharmaceutical ingredient having a high solubility in water that is used in steps (a) and/or (b) of the method for the preparation of cores is the same. More preferred the active pharmaceutical ingredient having a high solubility in water of the compacted API seeds, the active pharmaceutical ingredient having a high solubility in water that is used to prepare the spraying solution in step (2) of the method for the preparation of starter pellets and the active pharmaceutical ingredient having a high solubility in water that is used in steps (a) and/or (b) of the method for the preparation of cores is venlafaxine HCl.

In a further embodiment the present invention relates to the core obtainable by the method for the preparation of cores according to the present invention.

Preferably the cores have a particle size distribution of D10 of 400 μm to 1000 μm and/or a D50 of 750 μm to 1350 μm. More preferred the D10 is between 500 μm to 900 μm, such as between 600 μm to 800 μm, mostly preferred between 650 μm to 800 μm. More preferred the D50 is between 800 μm to 1300 μm, such as between 900 μm to 1200 μm, mostly preferred between 950 μm to 1150 μm.

Preferably the cores comprise 35% to 95% active pharmaceutical ingredient having a high solubility in water based on total weight of the core, such as between 45% to 95% based on total weight of the core, for example between 55% to 95% based on total weight of the core, mostly preferred between 65% to 95% based on total weight of the core.

Preferably the active pharmaceutical ingredient of the cores is an active pharmaceutical ingredient having a solubility in water at 25° C. of greater than or equal to 100 mg per gram of water. More preferred the active pharmaceutical ingredient of the cores is a venlafaxine salt, such as venlafaxine hydrogenmaleate anhydrate or venlafaxine HCl. Mostly preferred the active pharmaceutical ingredient of the cores is venlafaxine HCl.

In another embodiment the present invention relates to a pharmaceutical composition comprising the core of the present invention.

In yet another embodiment the present invention discloses a method for the preparation of film coated cores comprising the steps of
(I) preparing a coating dispersion and
(II) coating the cores according to the invention with the coating dispersion (I) to obtain film coated cores.

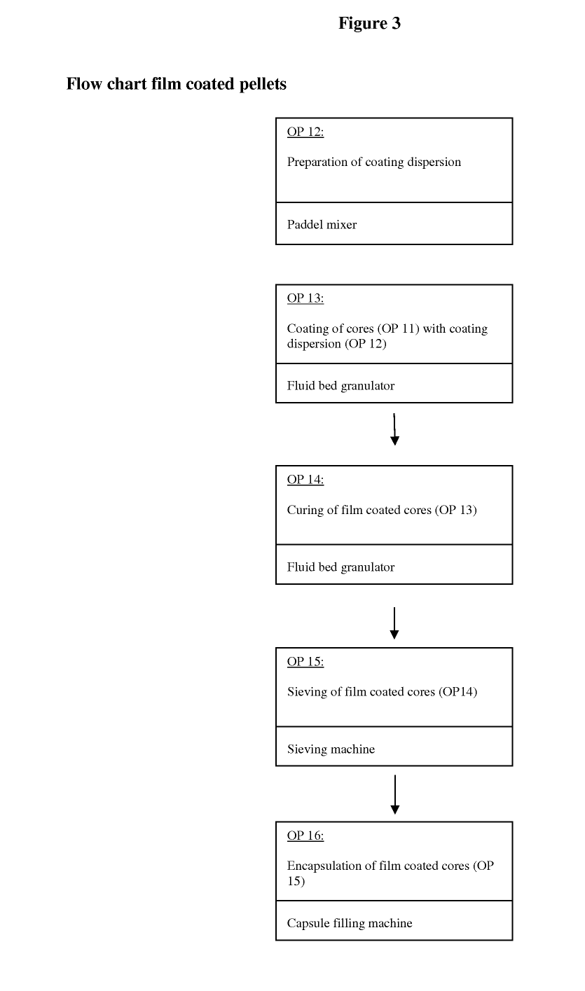

Optionally the method for the preparation of film coated cores may comprise further processing steps. For example the cores obtained in step (II) may further be cured and/or sieved. FIG. 3 shows an exemplary flow chart of the method to produce film coated cores.

Mostly preferred the method for the preparation of film coated cores comprises the steps of
(I) preparing a coating dispersion;
(II) coating the cores according to the invention with the coating dispersion (I) to obtain film coated cores;
(III) curing the film coated cores from step (II) and
(IV) sieving the film coated cores from step (III).

The film coated cores obtained in any one of steps (II), (III), and/or (IV) can be compressed into monolithic tablets, multi unit particulate tablets, multi unit pellet system (MUPS) or filled into capsules.

The coating dispersion in step (I) comprises one or more polymers. The polymers can either be polymers modifying the release of the active pharmaceutical ingredient or polymers not modifying the release of the active pharmaceutical ingredient. Preferably the polymers are modified release polymers, such as delayed release, prolonged release, sustained release, repeated action release, extended release and/or controlled release polymers. More preferred the modified release polymers are selected from the group consisting of cellulosic polymers, methacrylic acid polymers and waxes. Mostly preferred the modified release polymers are selected from the group consisting of ethyl cellulose, hydroxypropyl methylcellulose, hydroxypropyl cellulose, methylcellulose, carboxymethylcellulose, hydroxymethylcellulose, and hydroxyl-ethylcellulose, hydroxypropylmethyl phthalate, cellulose acetate phthalate, and cellulose acetate trimellitate. Particularly preferred, ethyl cellulose is used as modified release polymer. Ethyl cellulose can, for example, be used in the form of the commercially available Aquacoat® ECD system (FMC BioPolymer, approximately 24.5% to 29.5% ethyl cellulose in aqueous solution).

Preferred examples of polymers, which do not affect the release of the active pharmaceutical ingredient, can be those including polyvinyl alcohol, starch, modified starch, polyvinylpyrrolidone, sugars, sugar alcohols, aminoalkyl-methacrylate-copolymers, carbohydrates such as maltodextrins and polydextrose, mixtures of microcrystalline cellulose with carrageenan and the like.

The coating dispersion may comprise one or more pharmaceutically excipients and additives like e. g., film forming agent, anti-foam agent, filler, coloring agent, flavoring agent, perfume, sweetening agent, surface active agent, lubricant, stabilizing agent, antiadherent, pore former, plasticizer and the like, or mixtures thereof. Preferably the plasticizer is dibutyl sebacate.

The cores may be coated once or they may be coated more than once. If the cores are coated more than once, polymers belonging to the same or to a different group may be used in the different coating steps. For example the cores may first be coated with one or more polymers not affecting the release of the active pharmaceutical ingredient and in a second coating step using the above described method they may be coated with one or more polymers affecting the release of the active pharmaceutical ingredient or vice versa.

In a further embodiment the present invention relates to film coated cores obtainable by the method according to the present invention. The film coated cores comprise the cores according to the present invention and a coating film.

The coating film may have one or more layers. If the coating film has more than one layer, the different layers may comprise polymers belonging to the same or to a different group. For example the first layer may comprise one or more polymers not affecting the release of the active pharmaceutical ingredient and the second layer may comprise one or more polymers affecting the release of the active pharmaceutical ingredient or vice versa.

In another embodiment the present invention relates to a pharmaceutical composition comprising the film coated cores of the present invention. In still another embodiment the invention discloses pharmaceutical composition comprising the starter pellets, cores and/or film coated cores of the present invention. Preferably the pharmaceutical composition comprising the starter pellets, cores and/or film coated cores of the present invention is in form of pellets, cores and/or film coated cores filled into capsules.

The invention is now illustrated by way of examples which are not intended to be construed as limiting.

### EXAMPLES

### Example 1

### 1a Preparation of Compacted Venlafaxine HCl Seeds

The compaction parameters are shown below (Table 3).

TABLE 3
Parameters of compaction of venlafaxine HCl

| Parameter | Settings | |
|---|---|---|
| Compaction force | 16 | kN/cm |
| Gap width | 3.0 | mm |
| Rotation speed Compactor | 2.0 | rpm |
| Sieve size Granulator | 1.0 | mm |
| Rotation speed Granulator | 50 | rpm |
| Batch size | 85.0 | kg |
| Yield after compaction | 84.36 | kg |
| | 99.2% | |
| Sieve size for separation of | 0.4 | mm |
| fines | | |
| Yield after separation of fines | 52.40 | kg |

61.6%

For compaction a Roller compactor (Macro-Pactor M1126, Gerteis) was used. To obtain material with a tight particle size distribution, fines were removed by sieving, using a sieve size of 0.4 mm. An amount of 52.40 kg of venlafaxine particles between 0.4 and 1.0 mm was obtained, corresponding to 61.6% relating to the batch size of 85 kg.

### 1b Preparation of Starter Pellets

A fluid bed granulator with a volume of 50.0 L was chosen (Ventilus 50, Innojet). For manufacturing of the starter pellets the following excipients were used:

TABLE 4
Material used for preparation of starter pellets

| | g/batch | [%] |
|---|---|---|
| Material for powder mixture | | |
| compacted Venlafaxine HCl seeds | 3528.99 | 58.8 |
| Povidone 30 | 229.79 | 3.8 |
| Lactose D80 | 821.80 | 13.7 |
| Sodium hydrogen citrate | 360.64 | 6.0 |
| Silicon dioxide, colloidal | 57.45 | 1.0 |
| Microcrystalline cellulose | 1002.13 | 16.7 |
| Total | 6000.80 | 100.0 |
| Material for spraying solution | | |
| Venlafaxine HCl | 6000.00 | 30.0 |
| Purified water | 14000.00 | 70.0 |
| Total spraying solution | 20000.00 | 100.0 |

Compacted venlafaxine HCl, povidone 30, lactose D80, sodium hydrogen citrate, colloidal silicon dioxide and microcrystalline cellulose were filled into a fluid bed granulator.

For manufacturing of the spraying solution, venlafaxine HCl was dissolved in water until a clear solution with a solid content of 30% was obtained.

Starter pellets were obtained by spraying part of the venlafaxine HCl solution onto the powder mixture until most of the powder was bound. The following machine settings were used in the fluid bed granulator:

TABLE 5
Machine settings in fluid bed granulator
for production of starter pellets

| | | |
|---|---|---|
| | | Specification/ |
| Parameter | Value | Range |

| Air volume | 414-495 | m³/h | 200-700 | m³/h |
|---|---|---|---|---|
| Inlet air temperature | 60-68° | C. | 30-70° | C. |
| Spray rate | 171-187 | g/min | 100-250 | g/min |
| Spray pressure | 1.0-1.5 | bar | — | |
| Product temperature | 28-32° | C. | 20-45° | C. |
| Outlet air temperature | 34-38° | C. | 20-45° | C. |
| Amount of spray solution | 3511-3680 | g | | |

Afterwards the pellets were sieved through 0.7 mm and the fines were discarded. Pellets larger than 0.7 mm were used for the next manufacturing step. The yield before sieving fell within 6.923 and 7.101 kg, pellets larger than 0.7 mm amounted to 4.974 to 6.030 kg corresponding to 70.8-85.7%.

### 1c Preparation of Cores

For the powder feed the following substances were mixed in a tumbler mixer for 5 min at 17 rpm:

TABLE 6
Material used for powder feed

| Material | g/batch | [%] |
|---|---|---|
| venlafaxine HCl | 1805.32 | 45.1 |
| Povidone 30 | 153.19 | 3.8 |
| Lactose D80 | 1094.68 | 27.4 |
| Sodium hydrogen citrate | 240.43 | 6.0 |
| Silicon dioxide, colloidal | 38.30 | 1.0 |
| Microcrystalline cellulose | 668.09 | 16.7 |
| Total | 4000.01 | 100.0 |

One third of the powder feed was put to the starter pellets into the fluid bed granulator and spraying solution was sprayed onto it until fines were bound. To the resulting product one third of the powder feed was added and bound onto the particles by spraying the venlafaxine HCl solution until fines were bound. The procedure was repeated with the rest of the powder feed. The following machine settings were used in the fluid bed granulator:

TABLE 7
Machine settings in fluid bed granulator for powder feed

| | | |
|---|---|---|
| | | Specification/ |
| Parameter | Value | Range |

| Air volume | 455-527 | m³/h | 200-700 | m³/h |
|---|---|---|---|---|
| Inlet air temperature | 59-63° | C. | 30-75° | C. |
| Spray rate | 165-191 | g/min | 100-250 | g/min |
| Spray pressure | 1.0-1.5 | bar | — | |
| Product temperature | 31-33° | C. | 20-45° | C. |
| Outlet air temperature | 37-39° | C. | 20-45° | C. |
| Amount of spray solution | 5956-6120 | g | | |

Afterwards the cores were sieved through 0.7 mm and the fines were discarded. A yield of 10.833 to 11.817 kg was obtained before sieving, corresponding to 9.385 to 10.560 kg (84.8-90.1%) after sieving. Pellets larger than 0.7 mm were used for the smoothing step.

The venlafaxine HCl spraying solution was sprayed onto the pellets using the machine settings shown in Table 8.

TABLE 8
Machine settings in fluid bed granulator for smoothing

| | | |
|---|---|---|
| | | Specification/ |
| Parameter | Value | Range |

| Smoothing I | | | | |
|---|---|---|---|---|
| Air volume | 572-608 | m³/h | 200-700 | m³/h |
| Inlet air temperature | 57-63° | C. | 30-75° | C. |
| Spray rate | 173-182 | g/min | 100-250 | g/min |
| Spray pressure | 1.0-1.5 | bar | — | |
| Product temperature | 32-36° | C. | 20-45° | C. |
| Outlet air temperature | 36-42° | C. | 20-45° | C. |
| Amount of spray solution | 7700-7912 | g | | |
| Smoothing II | | | | |
| Air volume | 567-608 | m³/h | 200-700 | m³/h |
| Inlet air temperature | 49-51° | C. | 30-75° | C. |
| Spray rate | 78-85 | g/min | — | |
| Spray pressure | 1.0-1.5 | bar | — | |
| Product temperature | 36-38° | C. | 20-45° | C. |
| Outlet air temperature | 40-42° | C. | 20-45° | C. |
| Amount of spray solution | 2478-2763 | g | | |

Afterwards the cores were sieved through 0.7 mm and the fines were discarded. Additionally cores exceeding a diameter of 1.8 mm were removed by sieving. A yield of 12.515 to 13.668 kg was obtained before sieving, corresponding to 12.425 to 13.668 kg (98.5-99.3%) after sieving.

Table 9 shows results of in-process controls of eleven representative batches of cores:

TABLE 9
Results of in-process controls of cores

| | 120081 | 120082 | 120083 | 120084 | 120085 | 120086 | 120087 | 120088 | 120090 | 120102 | 120103 |
|---|---|---|---|---|---|---|---|---|---|---|---|
| Parameter | XU3P | XU3P | XU3P | XU3P | XU3P | XU3P | XU3P | XU3P | XU3P | XU3P | XU3P |
| Total Yield | 12.432 | 12.757 | 12.425 | 12.841 | 13.050 | 13.578 | 13.451 | 13.491 | 12.820 | 12.970 | 12.982 |
| 0.7-1.8 mm [kg] | | | | | | | | | | | |
| Total Yield | 77.7 | 79.7 | 77.7 | 80.3 | 81.6 | 84.9 | 84.1 | 84.3 | 80.1 | 81.1 | 81.1 |
| 0.7-1.8 mm [%] | | | | | | | | | | | |

Particle size distribution (after sieving)

| | | | | | | | | | | | |
|---|---|---|---|---|---|---|---|---|---|---|---|
| Dv(10) | 766 | 712 | 768 | 742 | 743 | 715 | 750 | 775 | 689 | 657 | 694 |
| Dv(50) | 1108 | 1071 | 1085 | 1046 | 1047 | 1037 | 1086 | 1087 | 1007 | 999 | 1011 |
| Dv(90) | 1549 | 1551 | 1498 | 1456 | 1457 | 1479 | 1528 | 1495 | 1454 | 1489 | 1456 |
| Residual moisture | 3.16 | 2.87 | 3.09 | 3.27 | 3.41 | 2.98 | 3.16 | 3.10 | 3.16 | 3.19 | 3.19 |
| [%] | | | | | | | | | | | |
| Abrasion* [%] | 0.0 | 0.0 | 0.0 | 0.0 | 0.0 | 0.0 | 0.0 | 0.0 | 0.0 | 0.0 | 0.0 |
| BET surface | 0.4393 | 0.4080 | 0.4975 | 0.5127 | 0.4675 | 0.5641 | 0.5684 | 0.5784 | 0.5543 | 0.4989 | 0.6120 |
| [m²/g] | | | | | | | | | | | |
| Content | 65.5 | 64.5 | 65.4 | 64.4 | 64.3 | 63.9 | 64.2 | 63.7 | 63.8 | 63.8 | 64.4 |
| Venlafaxine [%] | | | | | | | | | | | |

*Abrasion determined by putting 10 g of cores into a glass (fill grade about 20%) and mixing it in a planetary mixer and weighing the cores before and after mixing.

Film Coating with Ethylcellulose

Aquacoat, an aqueous ethylcellulose dispersion, was used as retardation principle in combination with dibutyl sebacate as plasticizer. The Aquacoat system consists of ethylcellulose, sodium lauryl sulfate and cetyl alcohol.

TABLE 10
Composition of Aquacoat dispersion with dibutyl sebacate

| Parameter | Value |
|---|---|
| Batch size used for coating [kg] | 12.0 |
| Dry polymer applied [%] | 14 |
| Composition of coating dispersion | |
| Aquacoat ECD 30 [g] | 6292.5 |
| Dibutyl sebacate [g] | 461.9 |
| Purified water [g] | 1292.2 |
| Total [g] | 8046.6 |
| Total solids [g] | 2349.6 |

The Aquacoat dispersion was prepared by adding dibutyl sebacate and stirring for more than 12 h to allow for a uniform distribution of the plasticizer in the colloidal ethylcellulose particles. Afterwards water was added and the dispersion was stirred for 10 min.

The following machine settings were used for coating in the fluid bed granulator:

TABLE 11
Machine settings in fluid bed granulator for coating

| | | |
|---|---|---|
| | | Specification/ |
| Parameter | Value | Range |

| Air volume | 690-711 | m³/h | 600-750 | m³/h |
|---|---|---|---|---|
| Inlet air temperature | 51-52° | C. | 45-65° | C. |
| Spray rate | 110-116 | g/min | 30-150 | g/min |
| Spray pressure | 1.5 | bar | — | |
| Product temperature | 36-39° | C. | 30-50° | C. |
| Outlet air temperature | 41-43° | C. | 30-50° | C. |

Immediately afterwards the pellets were cured in the fluid bed granulator for 2 h, cooled for 1 h with inlet air heating off and cured for 4 h again (machine parameters in 12).

TABLE 12
Machine settings in fluid bed granulator for curing

| | | |
|---|---|---|
| | | Specification/ |
| Parameter | Value | Range |

| Curing I | | | | |
|---|---|---|---|---|
| Curing time | 2 | h | 2 | h |
| Air volume | 700 | m³/h | 700 | m³/h |
| Inlet air temperature | 60° | C. | 60° | C. |
| Product temperature | 54-56° | C. | — | |
| Outlet air temperature | 55-57° | C. | — | |
| Cooling | | | | |
| Cooling time | 1 | h | 1 | h |
| Air volume | 700 | m³/h | 700 | m³/h |

| | | |
|---|---|---|
| Inlet air temperature | no heating of inlet | no heating of inlet |
| | air | air |
| Product temperature | gradually | — |
| | decreasing to room | |
| | temperature | |

| | | | | |
|---|---|---|---|---|
| Curing II | | | | |
| Curing time | 4 | h | 4 | h |
| Air volume | 700 | m³/h | 700 | m³/h |
| Inlet air temperature | 60° | C. | 60° | C. |

| | | | |
|---|---|---|---|
| Product temperature | 56° | C. | — |
| Outlet air temperature | 57° | C. | — |

Table 13 shows results of in-process controls of eleven representative batches of film coated cores:

TABLE 13
Results of in-process controls of film coated cores

| | 120081 | 120082 | 120083 | 120084 | 120085 | 120086 | 120087 | 120088 | 120090 | 120102 | 120103 |
|---|---|---|---|---|---|---|---|---|---|---|---|
| Parameter | XU3P | XU3P | XU3P | XU3P | XU3P | XU3P | XU3P | XU3P | XU3P | XU3P | XU3P |
| Total Yield | 14.111 | 13.810 | 13.860 | 13.810 | 13.980 | 13.932 | 13.880 | 13.816 | 13.920 | 13.890 | 14.001 |
| Yield <2.0 mm | 14.106 | 13.767 | 13.860 | 13.778 | 13.980 | 13.900 | 13.868 | 13.814 | 13.870 | 13.850 | 14.000 |
| [kg] | | | | | | | | | | | |
| Yield <2.0 mm | 98.3 | 95.9 | 96.6 | 96.0 | 97.4 | 96.9 | 96.6 | 96.3 | 96.7 | 96.5 | 97.6 |
| [%] | | | | | | | | | | | |

Particle size distribution (after sieving)

| | | | | | | | | | | | |
|---|---|---|---|---|---|---|---|---|---|---|---|
| Dv(10) | 952 | 924 | 971 | 951 | 964 | 972 | 980 | 783 | 739 | 902 | 838 |
| Dv(50) | 1269 | 1241 | 1286 | 1268 | 1279 | 1287 | 1295 | 1073 | 1013 | 1213 | 1141 |
| Dv(90) | 1617 | 1596 | 1627 | 1616 | 1622 | 1628 | 1633 | 1444 | 1386 | 1573 | 1523 |
| Residual | 0.30 | 0.29 | 0.37 | 0.33 | 0.36 | 0.30 | 0.36 | 0.36 | 0.29 | 0.32 | 0.36 |
| moisture [%] | | | | | | | | | | | |
| BET surface | 0.2283 | 0.0990 | 0.4004 | 0.3262 | 0.3131 | 0.1858 | 0.1852 | 0.2957 | 0.2458 | 0.2333 | 0.1926 |
| [m²/g] | | | | | | | | | | | |
| Content | 57.2 | 56.2 | 57.3 | 56.5 | 56.2 | 56.3 | 55.3 | 55.5 | 55.7 | 57.1 | 56.2 |
| Venlafaxine [%] | | | | | | | | | | | |
| t50% of | 5.2 | 5.5 | 3.3 | 3.8 | 3.8 | 5.5 | 4.8 | 5.2 | 4.3 | 3.8 | 5.2 |
| dissolution [h] | | | | | | | | | | | |

Particle size distribution after different cores manufacturing steps

| | Starter pellets | | | Powder feed | | | Smoothing | | |
|---|---|---|---|---|---|---|---|---|---|
| Manufacturing step | Dv(10) | Dv(50) | Dv(90) | Dv(10) | Dv(50) | Dv(90) | Dv(10) | Dv(50) | Dv(90) |
| 120083XU3P | 512 | 880 | 1433 | 681 | 1025 | 1512 | 768 | 1085 | 1498 |
| 120084XU3P | 574 | 959 | 1465 | 666 | 1026 | 1542 | 742 | 1046 | 1456 |
| 120085XU3P | 537 | 868 | 1362 | 707 | 1027 | 1473 | 743 | 1047 | 1457 |
| 120086XU3P | 598 | 959 | 1459 | 708 | 1027 | 1471 | 715 | 1037 | 1479 |
| 120087XU3P | 597 | 961 | 1465 | 699 | 1017 | 1463 | 750 | 1086 | 1528 |

Composition of film coated cores based on initial weight

| | 169.7 mg | 84.84 mg | 42.43 mg | |
|---|---|---|---|---|
| Ingredients | mg/capsule | mg/capsule | mg/capsule | [%] |
| venlafaxine HCl | 169.70 | 84.85 | 42.43 | 59.2 |
| Povidone | 5.73 | 2.87 | 1.43 | 2.0 |
| Lactose | 28.68 | 14.34 | 7.17 | 10.0 |
| Sodium hydrogen | 9.00 | 4.50 | 2.25 | 3.1 |
| citrate | | | | |
| Silicon dioxide, | 1.44 | 0.72 | 0.36 | 0.5 |
| colloidal | | | | |
| Microcrystalline | 25.01 | 12.51 | 6.25 | 8.7 |
| cellulose | | | | |
| Raw pellets | 239.56 | 119.78 | 59.89 | |
| Aquacoat ECD | 37.69 | 18.85 | 9.42 | 13.2 |
| Dibutyl sebacate | 9.22 | 4.61 | 2.31 | 3.2 |
| Film coated cores | 286.47 | 143.24 | 71.62 | 100.0 |

1. A method for the preparation of starter pellets comprising an active pharmaceutical ingredient having a solubility in water at 25° C. of between 100 mg per gram of water to 10 gram per gram of water comprising the steps of:
(1) preparing a powder mixture comprising compacted API seeds and one or more pharmaceutical excipients;
(2) preparing a spraying solution comprising an active pharmaceutical ingredient having a solubility in water at 25° C. between 100 mg per gram of water to 10 gram per gram of water and a solvent;
(3) spraying the solution of step (2) on the powder mixture obtained in step (1) to obtain starter pellets.

2-3. (canceled)

4. The method of claim 1, wherein the active pharmaceutical ingredient having a solubility in water at 25° C. of between 100 mg per gram of water to 10 gram per gram of water is a venlafaxine salt.

5. The method of claim 1, wherein the spraying solution comprises equal to or less than 30% (w/w) venlafaxine salt.

6. A starter pellet prepared by the method of claim 1.

7. A method for the preparation of cores comprising an active pharmaceutical ingredient having a solubility in water at 25° C. of between 100 mg per gram of water to 10 gram per gram of water comprising the steps of
(a) preparing a powder feed comprising an active pharmaceutical ingredient having a solubility in water at 25° C. of between 100 mg per gram of water to 10 gram per gram of water and one or more pharmaceutical excipients;
(b) preparing a spraying solution comprising an active pharmaceutical ingredient having a solubility in water at 25° C. of between 100 mg per gram of water to 10 gram per gram of water and a solvent;
(c) preparing a mixture of powder feed and starter pellets by combining one part of the powder feed from step (a) with the starter pellets according to the invention;
(d) spraying the solution of step (b) on the mixture from step (c);
(e) adding a further part of the powder feed from step (a) to the product of step (d);
(f) spraying the solution of step (b) on the product from step (e) and
(g) repeating steps (e) and (f) until all of the powder feed of step (a) is admixed with the starter pellets according to the invention to obtain cores.

8-9. (canceled)

10. The method of claim 7, wherein the active pharmaceutical ingredient having a solubility in water at 25° C. of between 100 mg per gram of water to 10 gram per gram of water is a venlafaxine salt.

11. The method of claim 10, wherein the spraying solution comprises equal to or less than 30% (w/w) venlafaxine salt.

12. A core prepared by the method of claim 7.

13. The core of claim 12, wherein said core has a particle size distribution of D10 of 400 to 1000 μm and/or a D50 of 750 to 1350 μm.

14. The core of claim 12, wherein the active pharmaceutical ingredient having a solubility in water at 25° C. of between 100 mg per gram of water to 10 gram per gram of water is a venlafaxine salt.

15. A pharmaceutical composition comprising the core of claim 12.

16. A method for the preparation of film coated cores comprising the steps of
(i) preparing a coating dispersion and
(ii) coating the core of claim 12 with the coating dispersion of step (i) to obtain film coated cores.

17. The method according to claim 16, wherein the coating dispersion comprises a modified release polymer.

18. The method according to of claim 16, wherein the coating dispersion comprises one or more modified release polymers selected from the group consisting of cellulosic polymers, methacrylic acid polymers, and waxes.

19. A film core prepared by the method of claim 16.

20. A pharmaceutical composition comprising one or more film coated cores of claim 19.

21. A method for the preparation of pure API seeds, said method comprising compacting a pure active pharmaceutical ingredient having a solubility in water at 25° C. of 100 mg per gram of water to 10 gram per gram of water.

22. The method according to claim 21 wherein the active pharmaceutical ingredient is venlaflaxine or desvenlaflaxine.

23. The method according to claim 21, wherein the active pharmaceutical ingredient is venlaflaxine HCl.

24. The method according to claim 21, wherein the compacting is carried out with a compaction force of 10-40 kN/cm.

25. A pure seed prepared by the method of claim 1.
